(19) United States
(12) Patent Application Publication
(10) Pub. No.: US 2006/0189684 A1
(43) Pub. Date: Aug. 24, 2006

(54) 3-Phenylfuran-2-one derivatives as cox-2 inhibitor

(76) Inventors: Juan Francisco Caturla Javaloyes, Barcelona (ES); Graham Warrellow, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/544,359

(22) PCT Filed: Feb. 12, 2004
(86) PCT No.: PCT/EP04/01296

§ 371 Date: Apr. 10, 2006

(30) Foreign Application Priority Data
Feb. 13, 2003 (ES) ES200300353

(51) Int. Cl. A61K 31/365; C07D 305/12

(52) U.S. Cl. 514/473; 549/323

(57) ABSTRACT

The present invention relates to 3-phenylfuran-2-ones of formula (I), processes for their preparation, pharmaceutical compositions containing them, and their medical uses.

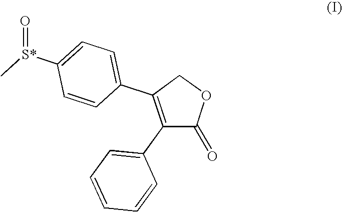

This invention relates to a new therapeutically useful 3-phenylfuran-2-one, to processes for their preparation, to pharmaceutical compositions containing them and to their use as medicaments.

It is known that non-selective inhibition of the enzyme cyclooxygenase (COX) prevents the overproduction of prostaglandins associated with inflammation, which are mediated by cyclooxygenase-2 (COX-2) but, at the same time, deprives tissues of basal levels of prostaglandins necessary for the health of certain tissues mediated largely by cyclooxygenase-1 (COX-1). Non steroidal anti-inflammatory drugs are non-selective inhibitors of COX and for that reason, have side effects of decreased renal blood flow, decreased platelet function, dyspepsia and gastric ulceration.

We have now found that certain 3-phenylfuran-2-ones selectively inhibit COX-2 in preference to COX-1 and are useful in the treatment of COX-2 mediated diseases and their symptoms, such as inflammation, pain, fever, and asthma, with fewer side effects.

Accordingly, the present invention provides a novel compound of formula (I)

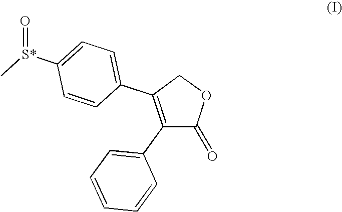

The compounds of formula (I) have a chiral center at the sulfur atom of the sulfinyl group, shown by an asterisk (*) in the formula, and consequently exist in the form of two different enantiomers. The two enantiomers and mixtures thereof including racemic mixtures are encompassed by the present invention. References to a compound of formula (I) in this specification, including the accompanying claims, unless otherwise specified, embrace each of the enantiomers and racemic and scalemic mixtures of the two enantiomers.

Other aspects of the present invention are: a) a process for the preparation of the compounds; b) pharmaceutical compositions comprising an effective amount of said compounds; c) the use of said compounds in the manufacture of a medicament for the treatment of diseases susceptible to amelioration by inhibition of the enzyme cyclooxygenase-2 (COX-2); and d) methods of treatment of diseases susceptible to amelioration by inhibition of the enzyme cyclooxygenase-2 (COX-2), which methods comprise the administration of the compounds of the invention to a subject in need of treatment.

Particular individual compounds of the invention are:
(R) 4-[4(methylsulfinyl)phenyl]-3-phenylfuran-2(5H)-one
(S) 4-[4(methylsulfinyl)phenyl]-3-phenylfuran-2(5H)-one

In another aspect the present invention encompasses a synthetic process for the preparation of the compounds of formula (I) which is depicted in Scheme 1 and involves the reaction of 2-bromo-1-[4-(methylthio)phenyl]ethanone (II) with phenylacetic acid (III) in the presence of a base to yield 4-[4-(methylthio)phenyl]-3-phenylfuran-2(5H)-one (IV) which is isolated and then oxidised to 4-[4(methylsulfinyl)phenyl]-3-phenylfuran-2(5H)-one (I).

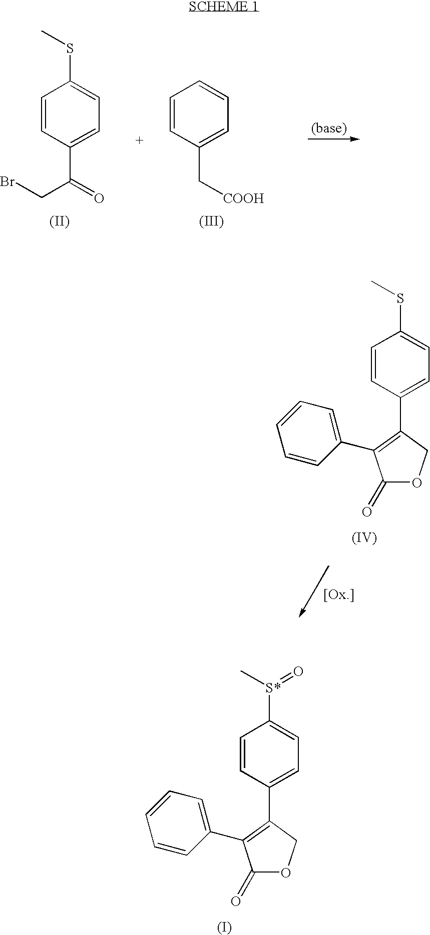

Following scheme (I) phenylacetic acid and a mixture of a base (e.g. potassium carbonate) and a crown ether are added to a suspension of 2-bromo-1-[4-(methylthio)phenyl]ethanone in a solvent (e.g. acetonitrile). The mixture is stirred at room temperature for 1 hour and 2 hours at reflux. After removal of the solvent, dichloromethane (400 ml) and saturated ammonium chloride (300 ml) are added to the residue. The organic layer is washed with water and brine, dried (Na₂SO₄) and concentrated in vacuo to give a residue, which was further purified to yield 4-[4-(Methylthio)phenyl)-3-phenylfuran-2(5H)-one.

According to the invention the compound of formula (I) is prepared by reaction of a compound of formula (IV) with an oxidising agent. The oxidation step can be made under non-stereo specific conditions or under stereo specific conditions. The oxidizing agent is preferably sodium metaperiodate when it is desired to obtain racemic mixtures of compounds or a mixture of titanium tetraisopropoxide, t-butyl hydroperoxide and either the (R, R) or the (S, S) forms of diethyl tartrate when it is desired to obtain mixtures of compounds of formula (I) enriched with compounds having a specific configuration at the sulfinyl chiral center. The reaction between the mercapto derivative of formula (IV) and the oxidising agent is preferably carried out in an organic solvent, preferably a chlorinated solvent or a mixture of chlorinated solvents and C₁-C₄ alcohols at a temperature of from −25° C. to 40° C. It is preferred that the chlorinated solvent is selected from the group consisting of 1,2-dichloroethane, methylene chloride, chloroform and mixtures thereof. The C₁-C₄ alcohol is preferably selected from methanol and ethanol. Preferred solvent systems are 1,2-dichloromethane or a mixture of methylene chloride with methanol or ethanol.

In the first case the mercapto compound of the previous step is dissolved in methanol and a solution of sodium metaperiodate is added dropwise at 0° C. and this mixture is stirred at this temperature for 2 hours and 3 days at room temperature. Then, the reaction mixture is poured into water, extracted with ethyl acetate, the organic solution washed with brine, dried (Na₂SO₄), and the solvent removed under reduced pressure. The residue, chromatographically purified, yields 4-[4-(Methylsulfinyl)phenyl)-3-phenylfuran-2(5H)-one (2.27 g, 81%) as an off-white solid.

In the second case t-butyl hydroperoxide in nonane and the mercapto compound of the previous step are added successively to a stirred solution of titanium tetraisopropoxide and an optically active diethyl tartrate (either the (R,R) or the (S,S) enantiomers) in dry 1,2-dichloroethane cooled to −20° C. The mixture is stirred at −20° C. for 5 h, then washed with a 5% aqueous solution of sodium sulfite (50 ml) and brine. The organic layer is dried (Na₂SO₄) and the solvent removed under reduced pressure. The residue after purification by flash chromatography yields an optically pure enantiomer of 4-[4-(Methylsulfinyl)phenyl)-3-phenylfuran-2(5H)-one obtained as an off-white solid.

Pharmacological Activity

The following biological tests and data further illustrate this invention.

### COX-1 and COX-2 Activities in Human Whole Blood

Fresh blood from healthy volunteers who had not taken any non-steroidal anti-inflammatory drugs for at least 7 days prior to blood extraction was collected in heparinized tubes (20 units of heparin per ml). For the COX-1 activity determination, 500 μl aliquots of blood were incubated with either 5 μl vehicle (dimethylsulphoxide) or 5 μl of a test compound solution for 24 h at 37° C. Calcium ionophore A23187 (25 μM) was added 20 min before the incubation was ended. Plasma was separated by centrifugation (10 min at 13000 rpm) and kept at −30° C. until TXB₂ levels were measured using an enzyme immunoassay kit (ELISA).

The effect of the compounds was evaluated by incubating each compound at five to six different concentrations with triplicate determinations. IC₅₀ values were obtained by non-linear regression using InPlot, GraphPad software on an IBM computer.

For the COX-2 activity determination, 500 μl aliquots of blood were incubated in the presence of LPS (10 μg/ml) for 24 h at 37° C. in order to induce the COX-2 expression (Patriagnani et al., J. Pharm. Exper. Ther. 271; 1705-1712 (1994)). Plasma was separated by centrifugation (10 min at 13000 rpm) and kept at −30° C. until PGE₂ levels were measured using an enzyme immunoassay kit (ELISA). The effects of inhibitors were studied by incubating each compound (5 μl aliquots) at five to six different concentrations with triplicate determinations in the presence of LPS for 24 hours. IC₅₀ values were obtained by non-linear regression using InPlot, GraphPad software on an IBM computer.

The results obtained from the biological assays are shown in Table 1 which shows the inhibition of COX-1 and COX-2 obtained with the racemic mixture of 4-[4-(methylsulfinyl)-phenyl]-3-phenylfuran-2(5H)-one.

TABLE I

| | COX-1 | COX-2 | |
|---|---|---|---|
| | IC₅₀ | IC₅₀ | Ratio |
| Example | μM | μM | COX-1/COX-2 |
| 4-[4(methylsulfinyl)phenyl]-3- | 33.4 | 4.93 | 6.8 |
| phenylfuran-2(5H)-one | | | |
| (racemate) | | | |
| 4-[4(methylsulfinyl)phenyl]-3- | 32.2 | 4.22 | 7.6 |
| phenylfuran-2(5H)-one | | | |
| (Enantiomer 1a) | | | |
| 4-[4(methylsulfinyl)phenyl]-3- | 37.8 | 2.10 | 18 |
| phenylfuran-2(5H)-one | | | |
| (Enantiomer 1b) | | | |

As shown in Table 1, the 3-phenylfuran-2-ones (I) are potent and selective COX-2 inhibitors. Thus the compounds of the invention are preferably selective inhibitors of mammalian COX-2, for example human COX-2.

The compounds of the invention also preferably have low inhibitory activity toward mammalian COX-1, for example human COX-1. Inhibitory activity can typically be measured by in vitro assays, for example as described above. The compounds of the present invention have also shown an unexpected pharmacokinetic profile.

Preferred compounds of the invention have an IC₅₀ value for COX-2 of less than 50 μM, preferably less than 10 μM more preferably less than 5 μM. Preferred compounds of the invention also have an IC₅₀ value for COX-1 of greater than 10 μM, preferably greater than 20 μM. As an indicator of selectivity for inhibition of COX-2 over COX-1, the ratio of COX-1/COX-2 IC₅₀ values is preferably greater than 10.

The present invention further provides a compound of formula (I) for use in a method of treatment of the human or animal body by therapy, in particular for the treatment of pain, fever or inflammation, to inhibit prostanoid-induced smooth muscle contraction or for the prevention or treatment of colorectal cancer or neurodegenerative diseases, for example, Alzheimer's disease.

The present invention further provides the use of a compound of formula (I) in the manufacture of a medicament for the treatment of pain, fever or inflammation, to inhibit prostanoid-induced smooth muscle contraction or for the prevention or treatment of colorectal cancer.

The compounds of formula (I) are useful for relief of pain, fever and inflammation of a variety of conditions including rheumatic fever, symptoms associated with influenza or other viral infections, common cold, low back and neck pain, dysmenorrhoea, headache, toothache, sprains and strains, myositis, neuralgia, synovitis, bursitis, tendinitis, injuries, following surgical and dental procedures and arthritis including rheumatoid arthritis, osteoarthritis, gouty arthritis, spondyloarthopathies, systemic lupus erythematosus and juvenile arthritis. They may also be used in the treatment of skin inflammation disorders such as psoriasis, eczema, burning and dermatitis. In addition, such compounds may be used for the prevention or treatment of colorectal cancer or neurodegenerative diseases, for example, Alzheimer's disease.

The compounds of formula (I) will also inhibit prostanoid-induced smooth muscle contraction and therefore may be used in the treatment of dysmenorrhoea, premature labour, asthma and bronchitis.

The compounds of formula (I) can be used as alternative to conventional non-steroidal anti-inflammatory drugs, particularly where such non-steroidal anti-inflammatory drugs may be contraindicated such as the treatment of patients with gastrointestinal disorders including peptic ulcers, gastritis, regional enteritis, ulcerative colitis, diverticulitis, Crohn's disease, inflammatory bowel syndrome and irritable bowl syndrome, gastrointestinal bleeding and coagulation disorders, kidney disease (e.g. impaired renal function), patients prior to surgery or taking anticoagulants, and patients susceptible to non-steroidal anti-inflammatory drugs induced asthma.

The compounds can further be used to treat inflammation in diseases such as vascular diseases, migraine headaches, periarteritis nodosa, thyroiditis, aplastic anaemia, Hodgkin's disease, scleroderma, type I diabetes, myasthenia gravis, sarcoidosis, nephrotic syndrome, Behcet's syndrome, polymyositis, hypersensitivity, conjunctivitis, gingivitis and myocardial ischaemia.

Compounds of the present invention are inhibitors of cyclooxygenase-2 enzyme and are thereby useful to treat the cyclooxygenase-2 mediated diseases enumerated above.

Accordingly, the compounds of the present invention and pharmaceutical compositions comprising such compounds may be used in a method of treatment of disorders of the human body which comprises administering to a patient requiring such treatment an effective amount of such compounds.

The present invention also provides pharmaceutical compositions, which comprise, as an active ingredient, at least a compound of formula (I) in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application.

Preferably the compositions are made up in a form suitable for oral, topical, nasal, inhalation, rectal, percutaneous or injectable administration.

The pharmaceutically acceptable excipients that are admixed with the active compound, to form the compositions of this invention are well known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules or liquid preparations, such as mixtures, elixirs, syrups or suspensions, all containing the compound of the invention; such preparations may be made by methods well-known in the art.

The diluents that may be used in the preparation of the compositions include those liquid and solid diluents that are compatible with the active ingredient, together with colouring or flavouring agents, if desired. Tablets or capsules may conveniently contain between 2 mg and 500 mg of active ingredient.

The liquid composition adapted for oral use may be in the form of solutions or suspensions. The solutions may be aqueous solutions of the active compound in association with, for example, sucrose to form a syrup. The suspensions may comprise an insoluble active compound of the invention in association with water, together with a suspending agent or flavouring agent.

Compositions for parenteral injection may be prepared from the compounds of the present invention which may or may not be freeze-dried and which may be dissolved in pyrogen free aqueous media or other appropriate parenteral injection fluid.

Effective doses are normally in the range of 10-600 mg of active ingredient per day. Daily dosage may be administered in one or more treatments, preferably from 1 to 4 treatments, per day.

The invention is illustrated by the following Preparation and Examples, which do not limit the scope of the invention in any way.

¹H Nuclear Magnetic Resonance Spectra were recorded on a Varian Gemini 300 spectrometer. Melting points were recorded using a Perkin Elmer DSC-7 apparatus. Optical rotations were determined on a Perkin Elmer 241MC Polarimeter. Enantiomeric purities were determined by capillary electrophoresis on an Agilent 3D (Agilent Technologies, Waldbronn, Germany), using a diode array detector and a capillary of melted silica (56 cm longitude, 50 micron internal diameter). The conditions used were the following: buffer (phosphoric acid 20 mM adjusted to pH 3.0 with triethanolamine, sulphobutylether cyclodextrin of substitution grade 7 (SBE-7CD), 10% acetonitrile); voltage (30 kV with negative polarity); temperature (20° C.); wavelength (200 nm (15 nm bandwidth) with a reference of 400 nm (80 nm bandwidth)).

### EXAMPLES

### Preparation 1

### 4-[4-(Methylthio)phenyl)-3-phenylfuran-2(5H)-one

To a suspension of 2-bromo-1-[4(methylthio)phenyl]ethanone (3.18 g, 13 mmol) in acetonitrile (70 ml) was added phenylacetic acid (1.77 g), 18-crown-6 (0.014 g) and potassium carbonate (3.22 g). The mixture was stirred at room temperature for 1 hour and 2 hours at reflux. Then, the solvent was removed under reduced pressure and dichloromethane (400 ml) and saturated ammonium chloride (300 ml) were added to the residue. The organic layer was washed with water and brine, dried (Na₂SO₄) and concentrated in vacuo to give a residue, which was purified by flash chromatography eluting with dichloromethane. 4-[4-(Methylthio)phenyl)-3-phenylfuran-2(5H)-one was obtained (2.31 g, 63%) as an orange solid.

δ (DMSO): 2.47 (s, 3H), 5.38 (s, 2H), 7.23-7.45 (m, 9H).

### Example 1

### 4-[4-(Methylsulfinyl)phenyl)-3-phenylfuran-2(6H)-one

To a solution of the title compound of Preparation 1 (1.80 g, 6.4 mmol) in methanol (31 ml) was added dropwise a solution of sodium metaperiodate (1.36 g) in water (15 ml) at 0° C. and this mixture was stirred at this temperature for 2 hours and 3 days at r.t. Then, the reaction was poured into water, extracted with ethyl acetate (3×100 ml), the organic solution washed with brine, dried (Na₂SO₄), and the solvent removed under reduced pressure. The residue was purified by flash chromatography and dichloromethane/ethyl acetate/ethanol/acetic acid (78/17/3/2) as eluent. 4-[4-(Methylsulfinyl)phenyl)-3-phenylfuran-2(5H)-one (2.27 g, 81%) was obtained as an off-white solid.

m.p.: 149-150° C.

δ (DMSO): 2.76 (s, 3H), 5.42 (s, 2H), 7.33-7.45 (m, 5H), 7.55 (d, J=8.4 Hz, 2H), 7.71 (d, J=8.4 Hz, 2H).

### Example 2

### (R)-4-[4-Methylsulfinyl)phenyl)-3-phenylfuran-2(5H)-one (Enantiomer 1a)

To a stirred solution of titanium tetraisopropoxide (1.05 ml, 3.5 mmol) and (R,R)-diethyl tartrate (2.45 ml, 14.2 mmol) in dry 1,2-dichloroethane (25 ml) cooled to −20° C. were added successively t-butyl hydroperoxide 5.5 M in nonane (1.29 ml, 7.1 mmol) and the title compound of Preparation 1 (1.0 g, 3.5 mmol). The mixture was stirred at −20° C. for 5 h, then washed with a 5% aqueous solution of sodium sulfite (50 ml) and brine. The organic layer was dried (Na₂SO₄) and the solvent removed under reduced pressure. The residue was purified by flash chromatography and ethyl acetate/methanol (95/5) as eluent. 4-[4-(Methylsulfinyl)phenyl)-3-phenylfuran-2(5H)-one (0.48 g, 45%, 100% ee) in the form of enantiomer 1a was obtained as an off-white solid.

[α]_{D}²²=+93.1 (c 0.25, MeOH)

m.p.: 149-150° C.

δ (DMSO): 2.76 (s, 3H), 5.42 (s, 2H), 7.33-7.45 (m, 5H), 7.55 (d, J=8.4 Hz, 2H), 7.71 (d, J=8.4 Hz, 2H).

### Example 3

### (S)-4-4-(Methylsulfinyl)phenyl)-3-phenylfuran-2(5H)-one (Enantiomer 1b)

Obtained in the form of enantiomer 1b as an off-white solid (63%, 93.4% ee) from the title compound of Preparation 1 and (S,S)-diethyl tartrate by the procedure described in Example 2.

[α]_{D}²²=−82.3 (c 0.25, MeOH)

m.p.: 149-150° C.

δ (DMSO): 2.76 (s, 3H), 5.42 (s, 2H), 7.33-7.45 (m, 5H), 7.55 (d, J=8.4 Hz, 2H), 7.71 (d, J=8.4 Hz, 2H).

### COMPOSITION EXAMPLES

### Composition Example 1

Preparation of tablets
Formulation:

| | | |
|---|---|---|
| Compound of the present invention | 5.0 | mg |
| Lactose | 113.6 | mg |
| Microcrystalline cellulose | 28.4 | mg |
| Light silicic anhydride | 1.5 | mg |
| Magnesium stearate | 1.5 | mg |

Using a mixer machine, 15 g of the compound of the present invention are mixed with 340.8 g of lactose and 85.2 g of microcrystalline cellulose. The mixture is subjected to compression moulding using a roller compactor to give a flake-like compressed material, The flake-like compressed material is pulverised using a hammer mill, and the pulverised material is screened through a 20 mesh screen. A 4.5 g portion of light silicic anhydride and 4.5 g of magnesium stearate are added to the screened material and mixed. The mixed product is subjected to a tablet making machine equipped with a die/punch system of 7.5 mm in diameter, thereby obtaining 3,000 tablets each having 150 mg in weight.

### Composition Example 2

Preparation of coated tablets
Formulation:

| | | |
|---|---|---|
| Compound of the present invention | 5.0 | mg |
| Lactose | 95.2 | mg |
| Corn starch | 40.8 | mg |
| Polyvinylpyrrolidone K25 | 7.5 | mg |
| Magnesium stearate | 1.5 | mg |
| Hydroxypropylcellulose | 2.3 | mg |
| Polyethylene glycol 6000 | 0.4 | mg |
| Titanium dioxide | 1.1 | mg |
| Purified talc | 0.7 | mg |

Using a fluidised bed granulating machine, 15 g of the compound of the present invention are mixed with 285.6 g of lactose and 122.4 g of corn starch. Separately, 22.5 g of polyvinylpyrrolidone is dissolved in 127.5 g of water to prepare a binding solution. Using a fluidised bed granulating machine, the binding solution is sprayed on the above mixture to give granulates. A 4.5 g portion of magnesium stearate is added to the obtained granulates and mixed. The obtained mixture is subjected to a tablet making machine equipped with a die/punch biconcave system of 6.5 mm in diameter, thereby obtaining 3,000 tablets, each having 150 mg in weight.

Separately, a coating solution is prepared by suspending 6.9 g of hydroxypropylmethyl-cellulose 2910, 1.2 g of polyethylene glycol 6000, 3.3 g of titanium dioxide and 2.1 g of purified talc in 72.6 g of water. Using a High Coated, the 3,000 tablets prepared above are coated with the coating solution to give film-coated tablets, each having 154.5 mg in weight.

### Composition Example 3

Preparation of capsules
Formulation:

| | | |
|---|---|---|
| Compound of the present invention | 5.0 | mg |
| Lactose monohydrate | 200 | mg |
| Colloidal silicon dioxide | 2 | mg |
| Corn starch | 20 | mg |
| Magnesium stearate | 4 | mg |

25 g of active compound, 1 Kg of lactose monohydrate, 10 g of colloidal silicon dioxide, 100 g of corn starch and 20 g of magnesium stearate are mixed. The mixture is sieved through a 60 mesh sieve, and then filled into 5,000 gelatine capsules.

### Composition Example 4

Preparation of a cream
Formulation:

| | |
|---|---|
| Compound of the present invention | 1% |
| Cetyl alcohol | 3% |
| Stearyl alcohol | 4% |
| Gliceryl monostearate | 4% |
| Sorbitan monostearate | 0.8% |
| Sorbitan monostearate POE | 0.8% |
| Liquid vaseline | 5% |
| Methylparaben | 0.18% |
| Propylparaben | 0.02% |
| Glycerine | 15% |
| Purified water csp. | 100% |

An oil-in-water emulsion cream is prepared with the ingredients listed above, using conventional methods.

1. A compound of formula (I): 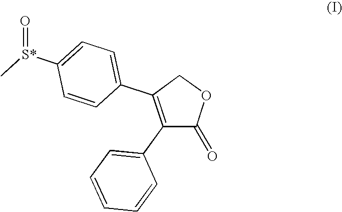
wherein the compound of formula (I) is in the form of any of the two different enantiomers;
or a racemic mixture of the enantiomers or a scalemic mixture of the enantiomers.

2. A compound according to claim 1, chosen from:
(R) 4-[4(methylsulfinyl)phenyl]-3-phenylfuran-2(5H)-one, and
(S) 4-[4(methylsulfinyl)phenyl]-3-phenylfuran-2(5H)-one.

3. A process for the preparation of a compound according to claim 1, comprising:
reacting a compound of formula (IV) 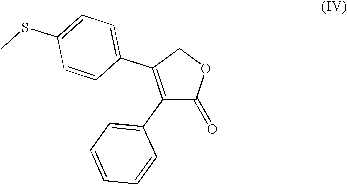
with an oxidizing agent.

4. A process according to claim 3 wherein the oxidizing agent is chosen from:
(a) sodium metaperiodate; and
(b) a mixture of titanium tetraisopropoxide, t-butyl hydroperoxide and either the (R,R) or the (S,S) form of diethyl tartrate.

5. A process according to claim 4, wherein the reaction takes place in at least one chlorinated solvent or in a mixture of at least one chlorinated solvent and at least one C₁-C₄ alcohol.

6. A process according to claim 5, wherein the at least one chlorinated solvent is chosen from 1,2-dichloroethane, methylene chloride, chloroform, and mixtures thereof.

7. A medicament comprising at least one compound as defined in claim 1.

8. (canceled)

9. A pharmaceutical composition comprising at least one compound according to claim 1 and at least one pharmaceutically acceptable diluent or carrier.

10. A pharmaceutical composition comprising at least one compound according to claim 2, and at least one pharmaceutically acceptable diluent or carrier.

11. (canceled)

12. A method for treating a subject afflicted with a pathological condition or disease susceptible to amelioration by inhibition of the enzyme cyclooxygenase-2 (COX-2), comprising administering to said subject an effective amount of a compound according to claim 1.

13. A method according to claim 12, wherein the pathological condion or disease is chosen from pain, fever, inflammation, prostanoid-induced smooth muscle contraction, colorectal cancer, and neurodegenerative diseases.

14. A method for treating a subject afflicted with a pathological condition or disease susceptible of amelioration by inhibition of the enzyme cyclooxygenase-2 (COX-2), comprising administering to the said subject an effective amount of a composition according to claim 10.

15. A method according to claim 14, wherein the pathological condition or disease is chosen from pain, fever, inflammation, prostanoid-induced smooth muscle contraction, colorectal cancer, and neurodegenerative diseases.
